(19) United States
(12) Patent Application Publication
(10) Pub. No.: US 2022/0370660 A1
(43) Pub. Date: Nov. 24, 2022

(54) DEVICE TO DISINFECT SPORTS BALLS USING ULTRAVIOLET LIGHT

(71) Applicant: Steribin LLC, St. George, UT (US)

(72) Inventors: Wayne Provost, Ivins, UT (US); Jonathan M. Cole, St. George, UT (US); Jeffrey Mel Glauser, Santa Clara, UT (US)

(73) Assignee: Steribin LLC, St. George, UT (US)

(21) Appl. No.: 17/746,565

(22) Filed: May 17, 2022

Related U.S. Application Data
(60) Provisional application No. 63/190,314, filed on May 19, 2021.

(51) Int. Cl. A61L 2/10

(52) U.S. Cl. CPC A61L 2/10; A61L 2202/11; A61L 2202/14; A61L 2202/16

(57) ABSTRACT

A disinfectant device can disinfect sports balls, such as basketballs, soccer balls, and volleyballs in an efficient manner, and may store the balls in a similar manner as current storage devices in the art. The disinfectant device for sports balls has a housing, a ball inlet, a plurality of rails, a plurality of disinfecting lights within the housing, and a ball outlet. The balls remain within the housing so as to be sufficiently exposed to UV light and thereby disinfected, and are advanced and released from the housing one at a time via an advancement mechanism.

### CROSS-REFERENCE TO RELATED APPLICATIONS

This application claims priority to U.S. Provisional Application Ser. No. 63/190,314, filed May 19, 2021, which is incorporated herein by reference.

### TECHNICAL FIELD

The present disclosure relates to a device for disinfecting items. More particularly, the present disclosure relates to a device for disinfecting sports balls.

### BACKGROUND

Infectious diseases commonly spread through the direct transfer of bacteria, viruses, or other microbes from contact with contaminated surfaces. Accordingly, disinfecting surfaces of items is important to maintaining health throughout the world. When surfaces are not disinfected to remove the viruses and bacteria thereon, people may become ill. One area where disinfecting items is important, but difficult, is sports equipment. In particular, sports balls come into contact with users' hands regularly, which increases the odds of spreading infectious diseases. However, disinfecting balls is difficult and time consuming.

Current methods in the prior art to disinfect sports balls are chemical sprays and UV light. Chemical sprays must be applied to the entire surface of the ball, which can be challenging. Further, chemicals can have a negative effect on the ball's surface, altering the playability of the ball. While there are a few products in the prior art that use UV light to disinfect sports balls, they have many deficiencies, including bulkiness, inadequate exposure to UV light on all surfaces, and inability to store the sports balls, among others.

Accordingly, there is a need for a disinfectant device for sports balls that can act as a storage rack, that disinfects the entire ball surface, and is inexpensive. The present disclosure seeks to solve these and other issues.

### SUMMARY OF EXAMPLE EMBODIMENTS

In some embodiments, a disinfectant device for sports balls comprises a housing, a ball inlet, a plurality of rails, a plurality of disinfecting lights within the housing, and a ball outlet. In some embodiments, an auger moves a ball within the housing to the outlet so as to release one or more balls from within the housing. The auger may be controlled by a controller so as to ensure adequate exposure within the housing. In some embodiments, the disinfectant device comprises casters for ease of transportation on a sport court or other location.

In some embodiments, the disinfectant device comprises one or more sensors for starting and stopping the auger (or other advancement mechanism) via a controller and motor.

### BRIEF DESCRIPTION OF THE DRAWINGS

FIG. 1 illustrates a front perspective view of a disinfectant device for sports balls;

FIG. 2 illustrates a front perspective view of a disinfectant device for sports balls with an access door removed;

FIG. 3 illustrates a front elevation view of a disinfectant device for sports balls with an access door removed;

FIG. 4 illustrates a top plan view of a disinfectant device for sports balls with a top panel removed;

FIG. 5 illustrates a top plan view of a disinfectant device for sports balls;

FIG. 6 illustrates a left side elevation view of a disinfectant device for sports balls; and

FIG. 7 illustrates a front perspective view of a disinfectant device for sports balls.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.). While ultraviolet (UV) light is used as an example throughout, it will be appreciated that any light, or electromagnetic wavelength of light, capable of destroying or inhibiting the growth of microorganisms is contemplated herein as a “disinfecting light.”

As previously discussed, there is a need for a disinfectant device for sports balls that can act as a storage rack, disinfects the entire ball, and is inexpensive. The present disclosure seeks to solve these and other issues.

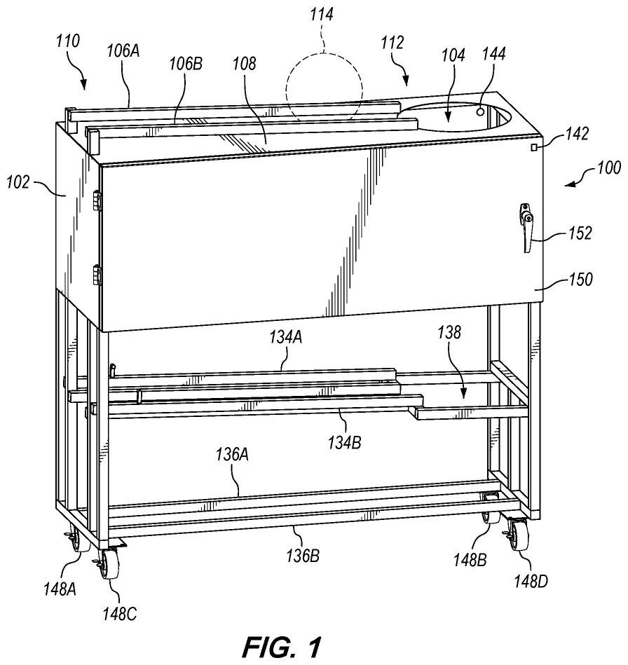

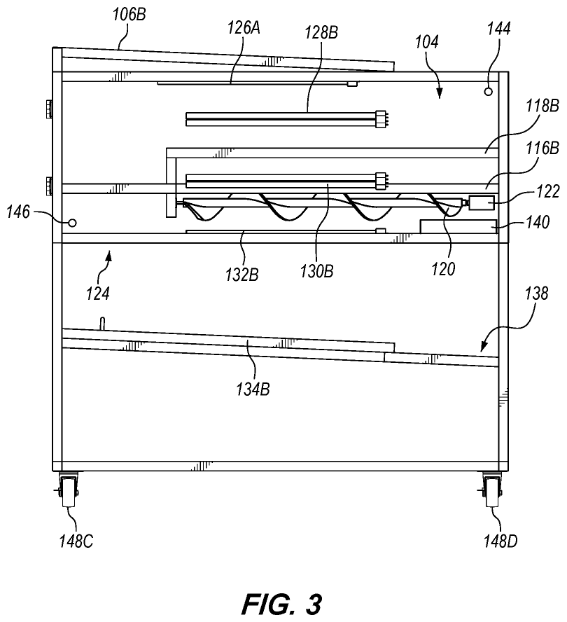

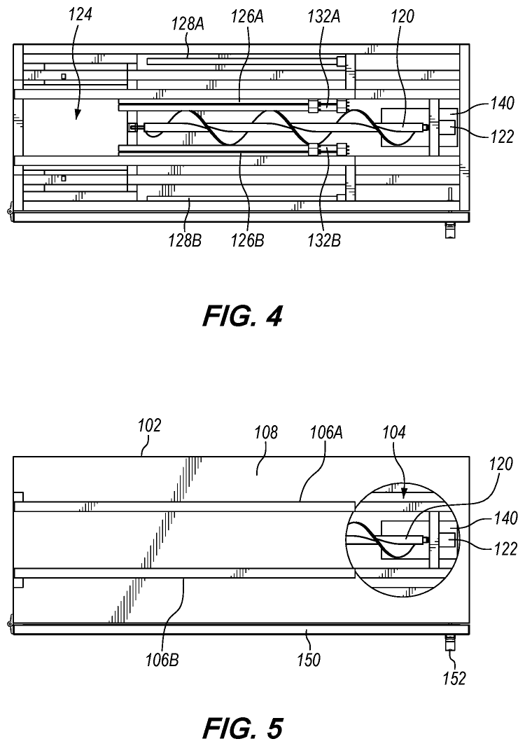

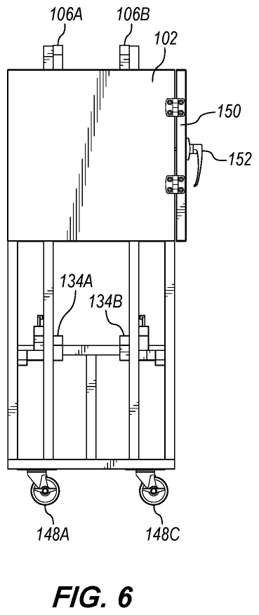

In some embodiments, as shown in FIGS. 1-6, a disinfectant device 100 for sports balls comprises a housing 102 and a ball inlet 104 sized so as to receive the desired sports balls. For example, the diameter of the ball inlet 104 may be sized complementary to a baseball, basketball, volleyball, bowling ball, etc. While sports balls are used as an example throughout, it will be appreciated that the invention is not so limited and any round object may be disinfected. A plurality of inlet rails 106A-B may be positioned on a top 108 of the housing 102 and sloped toward the inlet 104. In other words, a first end 110 of the rails 106A-B may have a first height in relation to the top 108 with the second end 112 may have a second height, which is less than the first height. As a result, a ball 114 placed on the rails 106A-B will roll from the first end 110 to the second end 112 where the ball 114 will then fall into the inlet 104.

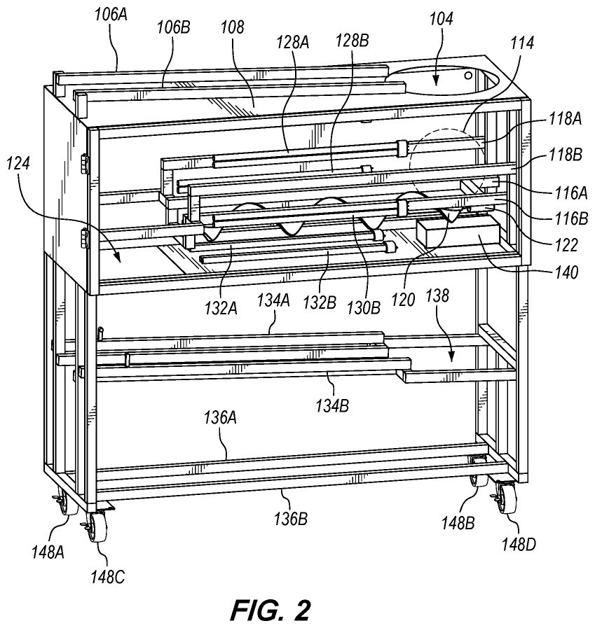

As best seen in FIGS. 2-4, once a ball 114 passes through the inlet, the ball 114 is received on a plurality of horizontal rails, such as a first horizontal rail 116A and a second horizontal rail 116B. The horizontal rails 116A-B are spaced apart from one other so as to receive a ball thereon with at least a portion of the ball 114 below the top surface of each horizontal rail 116A-B. A first cradle arm 118A and a second cradle arm 118B are positioned above the first and second horizontal rails 116A-B to prevent the ball 114 from rolling off the side of the horizontal rails 116A-B. The first and second cradle arm 118A-B run parallel to the horizontal rails 116A-B. In some embodiments, an auger 120 is positioned partially beneath, and interposed between, the horizontal rails 116A-B. The auger 120 is of sufficient height between the two horizontal rails 116A-B that the auger 120 prevents the ball 114 from freely rolling on the horizontal rails 116A-B. The auger 120 is controlled via a motor 122. In other words, when the motor 122 is activated, it actuates the auger 120, causing it to rotate and thereby move the ball 114 thereon from a first position beneath the inlet 104 to a second position (toward an outlet 124). It will be appreciated that the auger 120 need not progress the ball 114 from inlet 104 to the outlet 124 continuously, but may be stopped or otherwise pause intermittently.

As the ball 114 is within the housing 102, it is exposed to UV light from a plurality UV lights. For example, the disinfection device 100 may comprise top disinfecting lights 126A-B, upper disinfecting lights 128A-B, lower disinfecting lights 130B (only one visible, but appreciated that there are a plurality), and bottom disinfecting lights 132A-B. Accordingly, as the ball 114 moves along the horizontal rails 116A-B, the entire surface of the ball 114 is exposed to disinfecting light (e.g., UV light), thereby disinfecting it. It will be appreciated that the number, placement, size, and type of light used within the housing may vary without departing herefrom.

Once the ball 114 travels the entire length of the auger 120 and horizontal rails 116A-B, the ball 114 may exit the housing 102 through the outlet 124. As shown, the ball 114 may be received on a plurality of exit rails 134A-B. Exit rails 134A-B may be sloped so as to feed the ball 114 to a plurality of storage rails 136A-B via outlet 138. The balls 114 may be stored until retrieved by a user. It will be appreciated that the disinfectant device may be used for one ball or numerous balls and may house and store the balls both within the housing 102 and on the plurality of storage rails 136A-B.

In some embodiments, the auger 120 may be electronically controlled via a controller 140. The controller 140 (e.g., microcontroller) may be pre-programmed by a user to control the speed of the auger 120 via the motor 122 to ensure that each ball 114 that passes thereon is sufficiently exposed to the UV light so as to be disinfected. Additionally, the controller 140 may receive signals from one or more switches or sensors. For example, a user desiring to disinfect a ball 114, or retrieve a ball 114 from within the housing 102 may actuate a button or switch 142 (FIG. 1) on the exterior of the housing 102, which actuates the auger 120 via the motor 122 and the controller 140. When a ball has been disinfected, it will exit through the ball outlet 124 where it may be retrieved by a user.

In some embodiments, one or more sensors may actuate the motor 122 via the controller 140. For example, inlet sensor 144 may be an infrared sensor that, when interrupted, causes the controller 140 to activate the motor 122. In this example, a user may place a ball 114 on inlet rails 106A-B where the ball will roll into the ball inlet 104. As the ball 114 falls onto the horizontal rails 116A-B, it interrupts the infrared light of the inlet sensor 144, which triggers the controller 140 to power the motor 122 for a predetermined amount of time. In one example, the motor 122 operates until the ball 114 passes the length of the horizontal rails 116A-B so as to fall through the ball outlet 124. In some embodiments, the controller 140 may initiate the motor 122 long enough to advance any ball 114 on the horizontal rails 116A-B the distance of one ball. In this scenario, one or more balls 114 remain inside the housing 102 until fully advanced by the auger 120 by the addition of subsequent balls 114 or by a user overriding the inlet sensor 144, such as by actuating the button or switch 142.

In some embodiments, an outlet sensor 146 may stop the auger 120 via the controller 140 and motor 122. For example, a user may insert a ball 114 through the ball inlet 104. The auger 120 may activate via the inlet sensor 144 or via a button or switch 142 or other mechanism. The motor 122 may continue to operate until the outlet sensor 146 (e.g., infrared sensor) is triggered, signaling that a ball 114 has exited the housing 102. It may be the same ball 114 that was inserted or a ball 114 that was previously within the housing 102 awaiting exit. As a result, it is possible to dispense one ball 114 at a time. It will be appreciated that when set for single ball dispensing, an override switch may be activated if a user desires to run the auger 120 continuously. It will be appreciated that, to conserve energy, the plurality of UV lights 126A-132B may also be controlled via the controller 140. For example, in some embodiments, the UV lights only illuminate while the motor 122 is functioning to rotate the auger 120.

While only one row (i.e., horizontal rails 116A-B) of balls is shown within the housing 102, it will be appreciated that additional rows may be used so as to house and disinfect a greater number of balls. In other words, the size of the housing and number of rails 116A-B used therein may change without departing herefrom. Additionally, in some embodiments, a door may be used to close the ball outlet 124. The door may be hinged or pivotable so as to release one or more balls 114 from within the housing 102. The door may be manually operated, spring actuated, or electronically controlled via the controller 140. In some embodiments, the ball inlet may likewise comprise a door or flap so as to contain the UV light within the housing 102. In some embodiments, the disinfectant device 100 comprises casters 148A-D for ease of transportation on a sport court or other location. This allows the disinfectant device 100 to be maneuvered to a desired location by a user. While casters 148A-D are shown, they are not required.

In some embodiments, the disinfectant device 100 comprises an access door 150 for accessing the interior of the housing 102 for inspection or to replace parts, such as the lights. The access door may be hinged and operable using a handle 152. The handle 152 may be lockable so as to prevent unauthorized access to the internal components of the housing 102. In some embodiments, the disinfectant device may comprise a ball release. The ball release may comprise an auger, a retractable stopper, an actuatable door, or other mechanism, or any combination of mechanisms, for controlling the release of balls from within the housing. In some embodiments, a microcontroller controls the ball release to ensure a ball has sufficient exposure time within the housing prior to being released. In some embodiments, the ball release may combine a retractable stopper and an actuatable door.

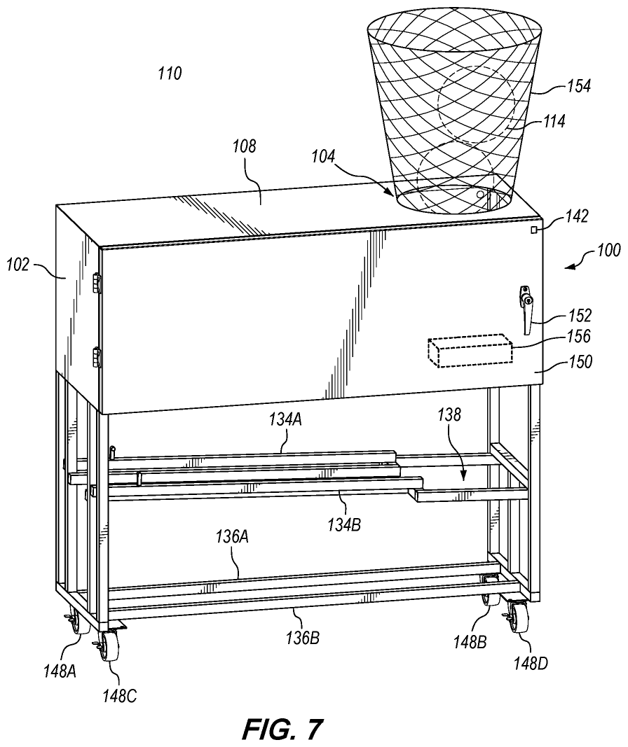

As shown in FIG. 7, the disinfectant device 100 for sports balls comprises a receiving cage 154 for receiving and/or storing balls 114 to be disinfected. In some embodiments, the receiving cage 154 may be funnel-shaped, so as to funnel balls 114 to the inlet 104. It will be appreciated that the receiving cage 154 may be in a variety of formfactors, including cuboid, cylindrical, triangular, etc. As a result, a user may place balls 114 in the receiving cage 154, which acts as a hopper. Balls 114 are then fed into the inlet 104 to be disinfected. The balls 114 may be simply gravity fed through the inlet 104, or may be controlled using a gate at the inlet 104. In other words, a plurality of balls 114 may be received within the receiving cage 154, with each ball 154 being fed, in succession, into the inlet 104 where they may then be disinfected using disinfecting lights within the housing 102. It will be appreciated that the components requiring electricity (e.g., disinfecting lights 126A-132B, auger 120 (i.e., motor 140), etc.) may receive the electricity through power cords and power outlets, or through one or more batteries 156. For example, a user may wheel the the disinfectant device 100 for sports balls onto a basketball court for use with basketballs for practice. A team may place a plurality of balls into the receiving cage 154, where each ball 114 is fed into the inlet 104 in succession. As each ball 114 progresses through the housing 102 via the auger 120, any ball 114 still in the receiving cage 154 may be received through the inlet 104 via gravity or via a gate. The disinfected balls 114 may then be retrieved from the storage rails 136A-B for play. Using batteries 156 allows the disinfectant device 100 for sports balls to be cordless so as to not pose risk of injury to players and so as to not require a power outlet nearby. When users are finished playing, they may once place the balls 114 in the hopper (receiving cage 154) where they may be disinfected for future play. The the disinfectant device 100 for sports balls may be wheeled off the court and stored while continuing to disinfect the balls 114 in the receiving cage 154. As discussed earlier, in some embodiments, one or more sensors 144, 146 may communicate with a controller 140, which may control the on/off status of the motor 122, disinfecting lights, and other components of the the disinfectant device 100 for sports balls. As a result, the the disinfectant device 100 for sports balls may be configured to power off when all balls 114 have entered the housing 102 and/or have been disinfected.

Further, although generally referred to herein as a “disinfecting device,” it is understood that a disinfecting device of the present disclosure may disinfect, sterilize, sanitize, or otherwise treat and clean the surface of a contaminated ball to achieve a lessened state or condition of contamination. Additionally, while an auger 120 is used as an example, it will be appreciated that other advancement devices may be used, which may include rollers, chains, belts, conveyors, or other mechanisms capable of horizontally advancing a ball from a first horizontal position to a second horizontal position. If a conveyor is used that inhibits light from the bottom, the speed and length of the conveyor may be adjusted to ensure the ball is sufficiently exposed to light on all sides. The speed with which a ball moves through the housing may be at a constant speed or at a variable speed. The sensors disclosed herein may be of any suitable type, including infrared, laser, mechanical switches, or others.

Housing 102 may include an exterior material having a first property or function, and an interior material having a second property or function that is different than the first property or function. For example, in at least one embodiment, the housing 102 may include an exterior material that is structurally rigid and opaque, and an interior material that is reflective. In some embodiments, the interior material of the housing 102 may include a coating applied to an inner surface of the exterior material of the housing 102. While illustrated with inlet rails 106A-B, it will be appreciated that they are not required. Balls 114 may be fed directly into the ball inlet 104 in top panel 108. In some embodiments, the ball inlet may be on the side of the housing, rather than the top panel 108. In some embodiments, a receiving cage 154 may be used to hold the balls 114 and feed them into the ball inlet 104. It will be appreciated that the cage may comprise solid materials, nets, or other materials suitable to funnel the balls 114 into the inlet 104.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A disinfectant device for sports balls, comprising:
a housing having a ball inlet and a ball outlet;
a first horizontal rail and a second horizontal rail within the housing extending from the ball inlet to the ball outlet, the first and second horizontal rails positioned so as to receive a ball thereon from the ball inlet;
a first cradle arm on a first side and a second cradle arm on a second side, the cradle arms positioned to prevent the ball from rolling off of a side of either the first or second horizontal rails;
a plurality of disinfecting lights positioned within the housing; and
an advancement mechanism actuatable via a motor.

2. The disinfectant device for sports balls of claim 1, wherein the advancement mechanism comprises an auger.

3. The disinfectant device for sports balls of claim 1, further comprising a controller configured to control the motor.

4. The disinfectant device for sports balls of claim 3, further comprising an inlet sensor.

5. The disinfectant device for sports balls of claim 3, further comprising an outlet sensor.

6. The disinfectant device for sports balls of claim 1, further comprising a first exit rail and a second exit rail each positioned beneath the housing to receive the ball from the ball outlet.

7. The disinfectant device for sports balls of claim 6, further comprising a plurality of storage rails positioned beneath the first and second exit rails.

8. The disinfectant device for sports balls of claim 1, further comprising a first inlet rail and a second inlet rail, each rail having a first height at a first end and second height lower than the first height at a second end, the second end positioned proximal to the ball inlet.

9. The disinfectant device for sports balls of claim 1, further comprising a plurality of casters.

10. A disinfectant device for sports balls, comprising:
a housing having a ball inlet and a ball outlet;
a first inlet rail and a second inlet rail coupled to a top panel of the housing, each rail having a first height at a first end and second height at a second end, the second height lower than the first height, the second end positioned proximal to the ball inlet;
a first horizontal rail and a second horizontal rail within the housing, the first and second rails positioned beneath the ball inlet and extending from the ball inlet to the ball outlet;
a first cradle arm on a first side and a second cradle arm on a second side, the cradle arms positioned to prevent a ball from rolling off of a side of either the first or second horizontal rails;
at least one top disinfecting light;
at least one upper disinfecting light;
at least one lower disinfecting;
at least one bottom disinfecting light;
an auger positioned partially beneath, and interposed between the first and second horizontal rails;
a motor to actuate the auger;
a controller configured to control the motor; and
at least one sensor for detecting the ball.

11. The disinfectant device for sports balls of claim 10, wherein the at least one sensor is an outlet sensor.

12. The disinfectant device for sports balls of claim 10, wherein the at least one sensor is an inlet sensor.

13. The disinfectant device for sports balls of claim 10, comprising an inlet sensor and an outlet sensor.

14. The disinfectant device for sports balls of claim 10, further comprising a first exit rail and a second exit rail, each positioned beneath the housing to receive the ball from the ball outlet.

15. The disinfectant device for sports balls of claim 14, further comprising a plurality of storage rails positioned beneath the first and second exit rails, the exit rails comprising an outlet configured to drop the ball onto the plurality of storage rails.

16. The disinfectant device for sports balls of claim 10, further comprising a plurality of casters for maneuvering the disinfectant device.

17. The disinfectant device for sports balls of claim 10, further comprising an access door.

18. The disinfectant device for sports balls of claim 17, wherein the access door is hingedly coupled to the housing.

19. The disinfectant device for sports balls of claim 10, further comprising a switch for controlling the motor.

20. The disinfectant device for sports balls of claim 10, further comprising a battery to power the motor and the at least one top disinfecting light, the at least one upper disinfecting light, the at least one lower disinfecting, and the at least one bottom disinfecting light.
